(19) United States
(12) Patent Application Publication
(10) Pub. No.: US 2009/0117054 A1
(43) Pub. Date: May 7, 2009

(54) SUBLINGUAL SPRAY FOR THE TREATMENT OF PAIN

(75) Inventors: Peter Crooks, Nicholasville, KY (US); Abeer Al-Ghananeem, Lexington, KY (US); Ahmad Malkawi, Lexington, KY (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 11/910,392

(22) PCT Filed: Mar. 29, 2006
(86) PCT No.: PCT/US06/11480

§ 371 Date: Aug. 15, 2008

Related U.S. Application Data
(60) Provisional application No. 60/666,060, filed on Mar. 29, 2005.

(51) Int. Cl. A61K 9/12; A61K 31/485; A61K 31/616; A61K 31/55; A61P 25/00

(52) U.S. Cl. 424/43; 514/282; 514/161; 514/217

(57) ABSTRACT

This invention relates to a spray for sublingual administration, used in the treatment and management of pain, especially acute pain. Also provided are methods of treatment and management of pain, a metered dosage system for administration of the spray, and combination therapies.

### FIELD OF THE INVENTION

This invention relates to a spray for sublingual administration, used in the treatment and management of pain, especially acute pain. Also provided are methods of treatment and management of pain, a metered dosage system for administration of the spray, and combination therapies.

### BACKGROUND

Adequate management of acute pain remains a serious medical problem. Acute pain often arises quickly, and lasts a relatively short time, as compared to long-term, chronic pain. However, while such pain is not chronic, it can be quite severe and often excruciating. Acute pain may result from disease, inflammation, injury to tissues, and other causes. This type of pain generally comes on suddenly, after a trauma or surgery for example, and may be further accompanied by anxiety, emotional distress and/or depression. The cause of acute pain can usually be diagnosed and treated, and the pain is self-limiting, i.e., it is confined to a given period of time and severity.

Current treatment options for acute pain (including oral, intravenous, and other formulations) do not provide the desired fast convenient relief from pain. These options often have poor bioavailability, titratability, and unpleasant side effects. Thus, new formulations and routes of administration are needed to provide fast relief from acute pain.

### SUMMARY OF THE INVENTION

The present invention provides a pharmaceutical composition comprising oxycodone or a pharmaceutically acceptable salt thereof and a pharmaceutically acceptable carrier, wherein the oxycodone or pharmaceutically acceptable salt thereof is provided in a form suitable for sublingual administration.

The present invention further provides a liquid spray formulation, comprising: (i) oxycodone or pharmaceutically acceptable salt or free base thereof, (ii) buffered water; and (iii) a polar organic solvent, wherein the polar organic solvent is present in an amount sufficient to enhance the solubility of the oxycodone free base or salt thereof in the water. The oxycodone may be present as the free base or salt. The formulation may be partially pressurized.

Preferably, the oxycodone or pharmaceutically acceptable salt or free base thereof, is present at a concentration of 0.1-10 mg/ml. Preferably, the polar organic solvent is an alcohol. The alcohol may include, but is not limited to, ethanol, propylene glycol, glycerol, polyethylene glycol and mixtures thereof. More preferably, the alcohol is ethanol. Preferably, the polar organic solvent is present in an amount of 3-50% w/w.

The formulation may be buffered. The formulation may further comprise a sweetener. The sweetener may be, but is not limited to, mannitol, saccharin or saccharin sodium. The formulation may further comprise a flavoring agent. Preferably, the flavoring agent is menthol. The formulation may further comprises a penetration enhancer. Preferably, the penetration enhancer is chitosan. The formulation may further comprise a mucoadherant. The mucoadherant may be, but is not limited to, chitosan, polyvinyl pyrrolidone, and gelatin.

Preferably, the present liquid spray formulation is suitable for sublingual administration. The formulation may further comprise another active ingredient including an opioid analgesic, a non-opioid analgesic, an antidepressant, a compound which reacts with the NMDA receptor, a compound which reacts with the nicotinic receptor, and pharmaceutically acceptable salts thereof.

The opioid analgesic may include, but is not limited to, codeine, morphine, hydromorphone, methadone, meperidine, levorphanol, fentanyl, meperidine, butorphanol, and pharmaceutically acceptable salts thereof. The non-opioid analgesic may include, but is not limited to, acetylsalicyclic acid, acetaminophen, ibuprofen, and pharmaceutically acceptable salts thereof. The antidepressant may include, but is not limited to, amitryptiline, desipramine, doxepin, imipramine, and pharmaceutically acceptable salts thereof. The compound which reacts with the NMDA receptor may include, but is not limited to, ketamine, norketamine, and pharmaceutically acceptable salts thereof. The compound which reacts with the nicotinic receptor may include, but is not limited to, nicotine, nornicotine, and pharmaceutically acceptable salts thereof.

The invention further provides a liquid spray formulation, comprising: oxycodone or pharmaceutically acceptable salt or free base thereof, in an amount of 0.01 mg/kg to about 10 mg/kg, wherein the liquid spray formulation is present in the amount of 100 mL.

The invention further provides a method of providing fast relief from pain, comprising administering to a subject in need thereof a pharmaceutically effective amount of oxycodone, by spraying the oxycodone onto the subject's sublingual mucosa. The pain may be acute. The acute pain may include post-operative pain, post-traumatic pain, or pain associated with cancer. Preferably, the oxycodone is in the form of oxycodone free base or hydrobromide salt, or any acceptable salt dissolved in an ethanolic solution.

The present invention further provides, a method of providing fast relief from acute pain comprising administering to a subject in need thereof a liquid spray formulation, comprising: (i) oxycodone or pharmaceutically acceptable salt or free base thereof, (ii) buffered water; and (iii) a polar organic solvent, wherein the polar organic solvent is present in an amount sufficient to enhance the solubility of the oxycodone free base or salt thereof in the water, by spraying the formulation onto the subject's sublingual mucosa. Preferably, the relief from pain is achieved within 20 minutes. More preferably, the relief from pain is achieved within 5 minutes.

The present invention further provides a metered dose dispensing system for the administration of a liquid spray formulation, comprising: (i) oxycodone or pharmaceutically acceptable salt or free base thereof, (ii) buffered water; and (iii) a polar organic solvent, wherein the polar organic solvent is present in an amount sufficient to enhance the solubility of the oxycodone free base or salt thereof in the water, comprising a sealed container fitted with a metering pump, an actuator and a channeling device. The metered dose dispensing system may contain a metering chamber which is adapted for dispensation with the container in the upright orientation, and wherein the metering chamber is in communication with the formulation by means of a dip-tube.

The present invention further provides a liquid spray formulation, comprising: (i) a compound that reacts with the same receptor as oxycodone or pharmaceutically acceptable salt or free base thereof, (ii) buffered water; and (iii) a polar organic solvent, wherein the said polar organic solvent is present in an amount sufficient to enhance the solubility of the compound of step (i), free base or salt thereof in the water.

### BRIEF DESCRIPTION OF THE FIGURES

FIG. 1 illustrates mean plasma oxycodone levels following sublingual administration of formulation 1 (pH 4), formulation 2 (pH 9), and intravenous (IV) administration at 100 μg oxycodone hydrochloride dose (n=3). All values show the mean±SEM (see Example 1).

FIG. 2 provides a graph showing the positive mode mass scan (the top chromatograph) and a DAD scan (bottom chromatograph) for oxycodone.

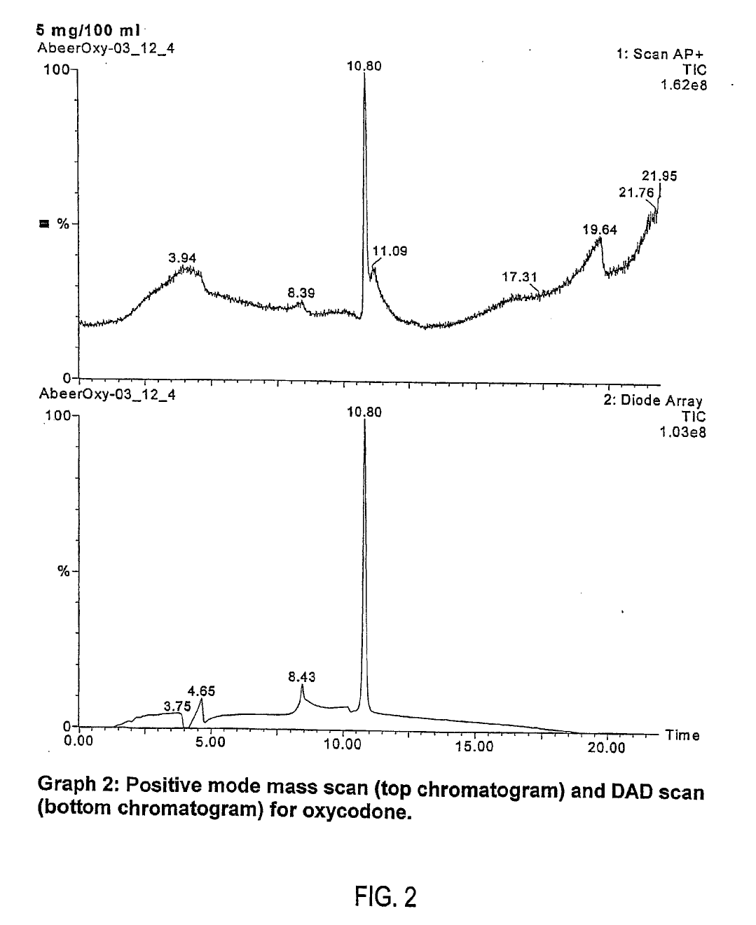

FIG. 3 illustrates a mass fragmentation scan on the positive mode for oxycodone.

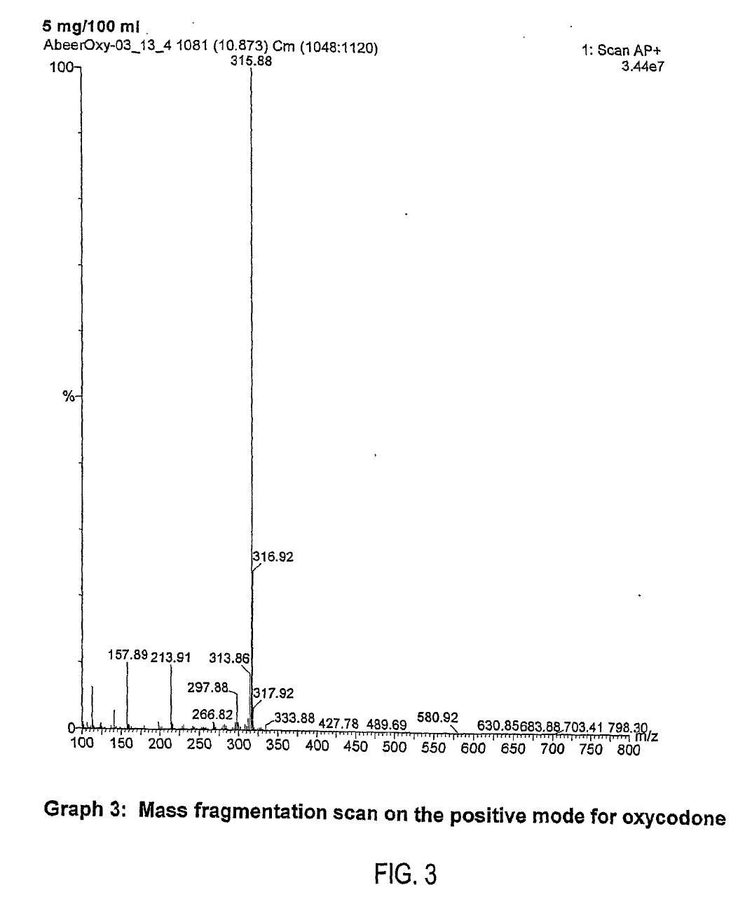

FIG. 4 illustrates the mass fragmentation of protonated oxycodone (OXC) (m/z at 316).

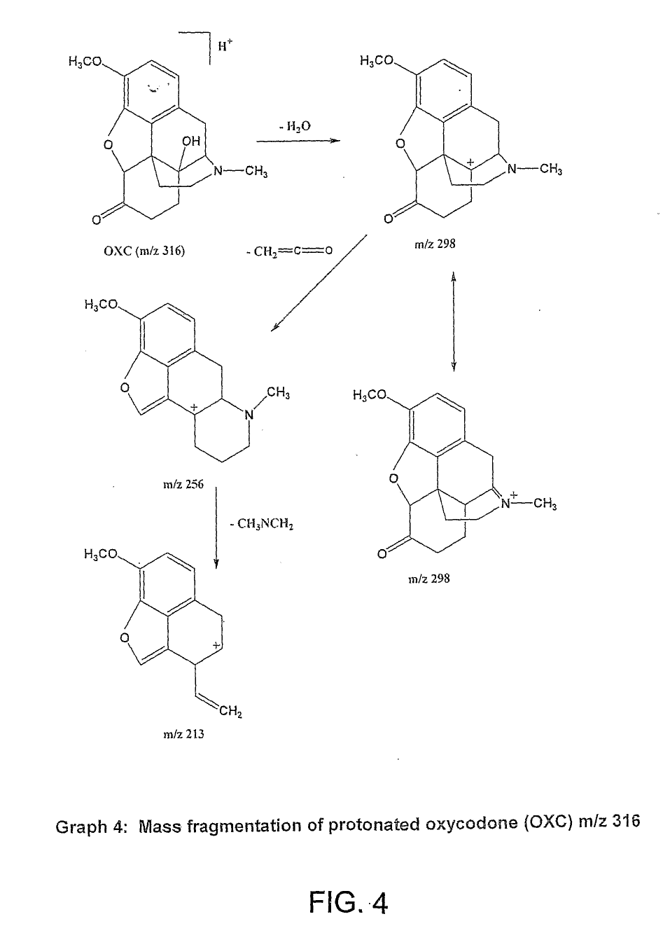

### DETAILED DESCRIPTION OF THE INVENTION

As described above, this invention provides a spray for sublingual administration for use in the treatment, management, inhibition and prevention of pain, especially acute pain. Also provided are methods of treatment, management, and prevention and of pain, combination therapies, and a metered dosage system for administration of the spray.

Prior to describing this invention in further detail, the following terms will first be defined.

### Definitions

In accordance with this detailed description, the following abbreviations and definitions apply. It must be noted that as used herein, the singular forms “a”, “an”, and “the” include plural referents unless the context clearly dictates otherwise. Thus, for example, reference to “compounds” includes a plurality of such compounds and reference to “the dosage” includes reference to one or more dosages and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless otherwise stated, the following terms used in the specification and claims have the meanings given below:

“Pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and claims includes both one and more than one such carrier.

“Treating” or “treatment” of pain includes:

(1) preventing the pain, i.e., causing the clinical symptoms of the pain not to develop in a mammal that may be exposed to or predisposed to the pain but does not yet experience or display symptoms of the pain,

(2) inhibiting and managing the pain, i.e., arresting or reducing the development of the pain or its cause(s), or

(3) relieving the pain, i.e., causing regression or cessation of the pain or its cause(s).

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Pharmaceutically acceptable salt” refers to pharmaceutically acceptable salts of oxycodone which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

“Optional” or “optionally” means that the subsequently described event or circumstance may, but need not, occur, and that the description includes instances where the event or circumstance occurs and instances in which it does not.

The term “fast” refers to the speed at which the present compositions and formulations provide relief from motion sickness and similar conditions. The term “fast” encompasses immediate relief up to about one hour, from the time the composition or formulation is administered.

The term “suitable for sublingual administration” refers to any mode of administration of a medicament to the tissue under the tongue. For example, a spray may be used.

The term “subject in need thereof” refers to any animal in need of relief from the symptoms of motion sickness, or the same or similar symptoms caused by any other disease or condition. Preferably, the subject is a mammal. More preferably, the subject is human.

The present invention provides a pharmaceutical composition comprising an analgesic compound, such as oxycodone, for sublingual administration. The present invention also provides a liquid formulation for administering to the sublingual mucosa (i.e., under the tongue) by a spray. This formulation preferably comprises an analgesic or acceptable salt or free base thereof, buffered water, and a polar organic solvent. The analgesic compound is preferably oxycodone, or another compound that reacts as an agonist with the same receptor as oxycodone.

Oxycodone (4,5-epoxy-14-hydroxy-3-methoxy-1 7-methyl-5a-morphinan-6-one) is a semi-synthetic opioid derived from the baine (George R. Lenz et al., Opiates, Academic, Orlando, 1987) that has been in clinical use since 1917 (Eija Kalso, Journal of Pain and Symptom Management. 2005;29:S47-S56). Oxycodone is usually indicated for severe acute postoperative pain, posttraumatic pain, and cancer pain (R. Poyhia et al, Journal of pain and symptom management. 1993;8:63-67).

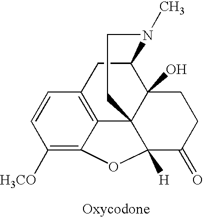

In the United States, oxycodone is available exclusively as oral formulations (D. S. Zhukovsky et al., Journal of pain and symptom management. 1999;18:53-55). However, often oral formulations are not the most effective or most convenient method of treatment for patients. Patients experiencing acute pain, such as from an operation or from trauma, may have difficulty in swallowing an oral dosage form. In addition, oral dosage forms do not provide the fast relief from the pain that is desired. Patients may have to wait a long time before the oral dosage is bioavailable and relief is experienced. Parenteral administration is also not always suitable, because of decreased venous access, and in the difficulty in administration.

Oxycodone has been administered to humans intramuscularly (R. Poyhia, et al., British Journal of Clinical Pharmacology. 1992;33:617-621), intranasally (A. Takala, et al., Acta Anaesthesiol Scand. 1997;41:309-312), orally using immediate release solutions, or as tablets and controlled-release tablets (J. Kaufmann, et al., Current Eye Research. 2004;28:271-275; G. R. Lauretti, et al., British Journal of Cancer. 2003;89:2027-2030; and R. Kaplan et al., Journal of clinical oncology: Official Journal of the American Society of Clinical Oncology. 1998;16:3230-3237), subcutaneously (I. Maddocks et al., Journal of pain and symptom management. 1996;12:182-189), and rectally (K. P. Leow, et al., Therapeutic drug monitoring. 1992;14:479-484). These routes are also not always desirable in the treatment of acute pain. For example, rectal administration results in greater variability compared with oral administration, and while the transdermal route may result in minimal pre-systemic hepatic elimination, it often exhibits a slow onset of action.

Drug delivery via the oral mucous membranes is a promising alternative to the known routes. The sublingual area of the oral cavity (the floor of the mouth and mucosa under the tongue) is more permeable than the buccal area (cheek), which in turn is more permeable than the palatal area (roof of the mouth) (Yuji Kurosaki, et al., Pharmaceutical Research. 1991;8:1297-1301). These differences in permeability are based on the relative thickness of the area, the blood supply, and degree of keratinization of these membranes. In addition to the differences in the permeability of the various mucous membranes, the extent of drug delivery is also affected by the physicochemical properties of the drug to be delivered. The sublingual route is an effective alternative to intravenous dosing for rapid delivery of drugs to the systemic circulation (D. Quintanar-Guerrero, et al., Biomaterials. 2001;22:957-961). Sublingual drug delivery by-passes gastrointestinal and hepatic pre-systemic elimination. It is also an acceptable and preferred form of drug delivery in patients with swallowing problems.

The high potency of oxycodone makes it suitable for sublingual delivery, due because the sublingual doses can be readily pre-prepared and administered using a metered dosage device. Sublingual administration is also easy and painless, especially when compared to intravenous routes. It can be self-administered with little trouble, and is especially useful in patients experiencing nausea, as well as in children. Sublingual drug administration is simple and cost-effective, and would be a good alternative for fast onset pain management.

### Spray Composition and Formulation

The present invention provides a pharmaceutical composition comprising an analgesic compound, such as oxycodone, provided for sublingual administration. The present invention also provides a liquid formulation for administering the analgesic compound to the sublingual mucosa (i.e., under the tongue) by a spray. This formulation preferably comprises oxycodone or acceptable salt or free base thereof, buffered water, and a polar organic solvent.

In general, the compounds of the subject invention will be administered in a therapeutically effective amount by any accepted sublingual mode of administration. Preferably, the formulation is administered as a liquid spray composition. Such compositions are prepared in a manner well known in the pharmaceutical art. Preferably, the spray is administered directly to the sublingual mucosa.

The oxycodone is present in the compositions and formulations in an amount sufficient to prevent, treat, relieve, manage, and/or inhibit pain, especially acute pain. The active compound is effective over a wide dosage range and is generally administered in a pharmaceutically or therapeutically effective amount. The therapeutic dosage of the compounds of the present invention will vary according to, for example, the particular use for which the treatment is made, the manner of administration of the compound, the health and condition of the patient, and the judgment of the prescribing physician. For sublingual administration by spray, the dose will typically be in the range of about 0.01 mg/ml to about 10 mg/ml, and preferably about 0.5 mg/ml to about 5 mg/ml. Effective doses can be readily extrapolated from dose-response curves derived from in vitro or animal model test systems.

The actual amount of the compound, i.e., oxycodone and acceptable salts and free bases thereof, will depend on a number of factors, such as the severity of the pain, the cause of the pain, the age and relative health of the subject, the potency of the compound used, the route and form of administration, and other factors.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture assays and animal studies can be used in future formulating of a range of dosage for use in humans and other animal patients. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The amount administered to the patient will vary depending upon what is being administered, the purpose of the administration, such as prophylaxis versus therapy, the state of the patient, the manner of administration, and the like. In therapeutic applications, compositions are administered to a patient already suffering from symptoms and/or a condition in an amount sufficient to cure or at least partially arrest the symptoms and complications. An amount adequate to accomplish this is defined as “therapeutically effective dose.” Amounts effective for this use will depend on the age, weight and general condition of the subject/patient, and the like.

The polar organic solvent is preferably present in an amount which will enhance the solubility of the oxycodone in water. Preferred organic solvents include, but are not limited to, alcohols, such as ethanol, propylene glycol, glycerol, polyethylene glycol and mixtures thereof. The polar organic solvent may be present in the formulation in an amount of about 3-50% w/w. The formulation may be buffered, as appropriate.

The compositions administered to a subject are in the form of pharmaceutical compositions. These compositions may be sterilized by conventional sterilization techniques, or may be sterile filtered. It will be understood that use of certain of the foregoing excipients, carriers, or stabilizers will result in the formation of pharmaceutical salts. When employed as pharmaceuticals, the compounds of the subject invention are usually administered in the form of pharmaceutical compositions. This invention also includes pharmaceutical compositions, which contain as the active ingredient, one or more of the compounds of the subject invention above, associated with one or more pharmaceutically acceptable carriers or excipients. The excipient employed is typically one suitable for administration to human subjects or other mammals. In making the compositions of this invention, the active ingredient is usually mixed with an excipient, diluted by an excipient. When the excipient serves as a diluent, it can be a solid, semi-solid, or liquid material, which acts as a vehicle, carrier or medium for the active ingredient.

The compositions of the invention can be formulated so as to provide quick, sustained or delayed release of the active ingredient after administration to the patient by employing procedures known in the art. The oxycodone formulation may further comprise a sweetened, such a mannitol, saccharin, and saccharin sodium. The formulation may further comprise a flavoring agent, such as menthol.

To assist in the speed of efficacy and bioavailability, the formulation may also comprise a penetration enhancer. Preferably, the penetration enhancer is chitosan. When formulated with a penetration enhancer such as chitosan, the bioavailabliliy of the formulation can reach 90% or greater. The formulation may also comprise a mucoadherant to increase the residence time on the mucosa; including chitosan, polyvinyl pyrrolidone, or gelatin.

The formulation may further comprise a moisturizing agent, such as propylene glycol, or polyethylene glycol. The formulation may further comprise a preservative such as sodium metabisulphite, benzalkonium chloride, or ethanol. The formulation may further comprise an antioxidant, such as butylated hydroxyltoluene, ascorbic acid, alkyl gallates, or tocopherols. The formulation may further comprise an ionic or nonionic surfactant, such as sodium lauryl sulfate, or sorbitan esters.

The liquid forms in which the compositions of the present invention may be incorporated for administration by spray include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles. A spray formulation may be prepared by methods well known in the art.

When administered as a spray for sublingual administration, the oxycodone is preferably in the form of the oxycodone free base or hydrobromide salt, or any acceptable salt dissolved in an ethanolic solution.

According to one aspect of the invention, the compound may be administered alone, or in combination with any other medicament. Thus, the spray formulation may comprise oxycodone, or compound acting on the same receptor as oxycodone, in combination with another, second active ingredient in the same formulation. The second active ingredient may include, but is not limited to, an opioid analgesic, a non-opioid analgesic, an antidepressant, a compound which reacts with the NMDA receptor, a compound which reacts with the nicotinic receptor, and pharmaceutically acceptable salts thereof. These active ingredients often have a beneficial synergistic effect when administered with oxycodone.

The opioid analgesic may include, but is not limited to, codeine, morphine, hydromorphone, methadone, meperidine, levorphanol, fentanyl, meperidine, butorphanol, and pharmaceutically acceptable salts thereof. The non-opioid analgesic may include, but is not limited to, acetylsalicyclic acid, acetaminophen, ibuprofen, and pharmaceutically acceptable salts thereof.

Patients experiencing pain, including acute pain, often suffer further from depression, emotional debilitation, and other mental distress. Patients addicted to drugs and geriatric patients also often experience acute pain. Thus, the present formulation may also comprise an antidepressant or other drug indicated for the treatment of depression, anxiety, other emotional distress or drug addiction. The antidepressant may include, but is not limited to, amitryptiline, desipramine, doxepin, imipramine, and pharmaceutically acceptable salts thereof.

The compound which reacts with the NMDA receptor may include, but is not limited to, ketamine, norketamine, and pharmaceutically acceptable salts thereof. Ketamine is a non-competitive NMDA receptor antagonist. Because ketamine is known to suppresses breathing and circulatory function much less so than most other available anaesthetics, ketamine is often used as a first-line anaesthetic for victims with unknown medical history, such as a victim of a car accident or other trauma. The compound which reacts with the nicotinic receptor may include, but is not limited to, nicotine, nornicotine, and pharmaceutically acceptable salts thereof.

When administered in combination, the compounds may be administered in the same formulation as these other compounds as shown, or in a separate formulation. When administered in combination, the spray may be administered prior to, following, or concurrently with the other compounds and compositions.

Suitable methods and formulations for use in the present invention are found in REMINGTON'S PHARMACEUTICAL SCIENCES, Mace Publishing Company, Philadelphia, Pa., 17th ed. (1985).

### Methods of Treatment, Pain Management and Prevention

The present invention provides methods of treating, managing and/or preventing pain, especially acute pain. Acute pain may be caused by any source, including surger, trauma, migraine headache, and dental pain, and may be accompanied by anxiety, emotional distress and/or depression.

The formulation is preferably administered as a spray. Preferably, the spray is administered directly to the sublingual mucosa, i.e., the formulation is sprayed directly onto the tissue under the patient's tongue. By administering the oxycodone directly to the sublingual mucosa, the patient can experience fast and even immediate relief, while still maintaining a high level of bioavailability. A patient suffering from these symptoms can feel relief within 1-5 minutes, with a maximum concentration of the drug being reached within 20 minutes or faster. Thus, someone suffering from acute pain can experience immediate relief. A patient experiencing this type of pain cannot anticipate the advent of the pain in advance and take a prophylactic dosage of pain medication, or is not in a position to safely do so.

### Metered Dosage System

The present invention further provides a device and system for administering the spray. Such a system can include a metered dose dispensing system, providing a convenient way to confirm that each spray dose is identical in amount. The metered dosage system may comprise a sealed container, which is fitted with a metering pump, an actuator and a channeling device. The metered dosage system may further contain a metering chamber adapted for dispensation with the container in the upright orientation. The metering chamber would be placed in communication with the formulation by means of a dip-tube.

Metered dosage systems well known in the art may be used. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The following example is offered to illustrate this invention and is not to be construed in any way as limiting the scope of this invention.

### EXAMPLES

### Example 1

A study was performed to develop a sublingual spray drug delivery formulation of oxycodone for acute pain management, using rabbit as the animal model, and to evaluate the effect of formulation pH on the sublingual absorption of oxycodone. A rabbit animal model was used to investigate sublingual oxycodone absorption because the histology and drug transport properties of rabbit's sublingual mucosa are similar to those of human.

Oxycodone hydrochloride, ethanol, propylene glycol, citric acid, dibasic sodium phosphate, and formic acid were obtained from Sigma-Aldrich Chemical Co. (St Louis, USA). Hydrochloric acid, purified water USP, chloroform, and HPLC grade acetonitrile were obtained from Fisher Scientific (Pittsburgh, Pa.). Water for HPLC use was passed through a reverse osmosis system (Milli-Q® Reagent Water System) before use. Isoflourane gas for anesthesia was provided by VMC Anesthesia (Ohmeda Waukesha, Wis.). Siliconized microcentrifuge tubes, vials, and tips were purchased from Fisher Scientific (Fair Lawn, N.J.). Saline (0.9%, injectable) was purchased from Baxter Healthcare Corporation (Deerfield, Ill.). Heparinized caraway capillary tubes were purchased from Baxter Healthcare Corporation (McGraw Park, Ill.). Tuberculin Slip tip Sterile Catheters were purchased from J&J Medical (New Brunswick, N.J.).

Male New Zealand albino rabbits weighing between 3.0-3.5 kg (Myrtle's Rabbitry Inc., Thompson Station, Tenn.) were used. The animal work was conducted at the University of Kentucky Chandler Medical Center, Division of Laboratory Animal Resources (DLAR). All research and testing activities related to this work were reviewed and approved by the Institutional Animal Care and Use Committee (IACUC) prior to the initiation of this research, and during its execution.

Following introduction of anesthesia (isoflourane general anesthetic gas) a catheter was placed in the marginal ear vein of the rabbit for blood sample collection. For sublingual spray administration, the oxycodone dose (100 μg) of either formulation solution 1 (pH 4) or 2 (pH 9) (Table 1) was applied to the sublingual mucosa of the rabbit through a spray bottle (n=3 rabbits/formulation per route). A separate in vitro spray weight evaluation was performed for the spray bottle before dosing. The spray bottle was hand-filled with 2.5 mL deionized water and actuated 10 times for priming before obtaining spray weight data. After priming, net spray weight measurements were taken for 10 consecutive actuations. Target delivery weight for each single spray was around 0.1 g. For intravenous administration, oxycodone hydrochloride aqueous solution was utilized; a sterile drug solution was prepared by filtration (double 0.22 μm filters), and a dose of 100 μg oxycodone injected into the marginal ear vein cannula followed by a 0.1 mL flush with 10% (v/v) heparin/normal saline solution to keep the cannula patent.

TABLE 1
Active and inactive contents of formulation solutions
1 (pH 4) and formulation solution 2 (pH 9).

| | Quantity/100 ml | | |
|---|---|---|---|
| Active | | Formulation 1 | Formulation 2 |
| ingredient/excipient | | (pH 4) | (pH 9) |

| | | | | |
|---|---|---|---|---|
| Oxycodone HCl | 100 | mg | 100 | mg |
| Absolute alcohol | 30 | ml | 35 | ml |
| Propylene glycol | 8 | ml | 8 | ml |
| Phosphate/Citrate | 100.0 | qs | 100.0 | qs |
| buffer | | | | |

Aliquot parts of 1 mL blood samples were collected as follows: at baseline, before oxycodone administration; immediately after oxycodone administration; and subsequently at 5, 10, 20, 45, 60, and 120 minutes following oxycodone administration. Blood samples were injected into pre-heparinized tubes and immediately placed on ice. Plasma was separated by centrifugation at 3000 rpm for 10 minutes, placed in polypropylene tubes, and frozen at −20° C. until the time of analysis.

Protein in rabbit plasma samples was precipitated with 600 μL acetonitrile, in 1.5 mL polypropylene test tubes. The samples were vortexed for 20 seconds and centrifuged at 14,000rpm for 10 minutes. 500 μL aliquot parts of the resulting supernatant were directly transferred to autosampler vials containing low volume inserts, evaporated to dryness with nitrogen gas at ambient temperature, and then reconstituted with 100 μL acetonitrile. 50 μl aliquot parts of this final solution were injected onto the HPLC-MS system.

Chromatography was performed on an Agilent Zorbax® Eclipse C₈ (4.6 mm×150 mm, 5 μm) column with a mobile phase consisting of mobile phases (A) water with 0.1% formic acid, and (B) acetonitrile with gradient elution (Table 2).

TABLE 2
Gradient elution time-table program in the HPLC-MS
system using mobile phase A (water with 0.1% formic
acid) and mobile phase B (acetonitrile)

| Time (minutes) | A % | B % |
|---|---|---|
| 0.0 | 100.0 | 0.0 |
| 2.0 | 100.0 | 0.0 |
| 15.0 | 0.0 | 100.0 |
| 18.0 | 0.0 | 100.0 |
| 20.0 | 100.0 | 0.0 |
| 22.0 | 100.0 | 0.0 |

The flow-rate was set at 0.5 mL/min. The LC-MS system consisted of a Waters 2690 HPLC pump (Waters, Milford, Mass.), a Waters 2695 autosampler, and a Micromass ZQ detector (Waters, Milford, Mass.) which utilized electrospray ionization (ESI). Selected ion monitoring (SIM) was performed in the positive mode for oxycodone, M+=316 m/z (dwell time 0.3 s), the capillary voltage was 4.5 kV and the cone voltage was 30 V. The source block and desolvation temperatures were 120 and 250° C., respectively. Nitrogen was used as the nebulization and drying gas at flow rates of 50 and 450 L/h, respectively. Calibration curves were constructed using a linear regression of the drug peak area versus nominal drug concentrations. The method was validated over the concentration range used, and found to be satisfactory for the determination of oxycodone in rabbit plasma over the concentration range of 5-200 ng/ml. The limit of quantification (LOQ) was established at 5 ng/ml. MS control and spectral processing were performed using MassLynx™ software, version 3.5.

Concentration-time profiles of oxycodone after IV and sublingual administration of formulations 1 and 2 were evaluated by a non-compartmental model (WinNonlin Professional, version 4.1, Pharsight Corporation, Mountain view, Calif.). The pharmacokinetic parameters, such as terminal elimination half life (t_{1/2}), area under the curve from 0 to infinity, AUC_{0-∞} were estimated using this software.

After a single dose, maximum plasma concentration (Cₘₐₓ), and time to reach maximum concentration (Tₘₐₓ) were also determined. The absolute bioavailability of the sublingual formulation was calculated by Equation (1):

$$F = \frac{AUC SL}{AUC IV} \times \frac{Dose IV}{Dose SL} \times 100 \qquad ( 1 )$$

Where F is the percent absolute bioavailability, and AUC_{SL}, AUC_{IV}, Dose_{IV}, Dose_{SL} are the area under the curve and corresponding dose for the sublingual and intravenous administrations, respectively.

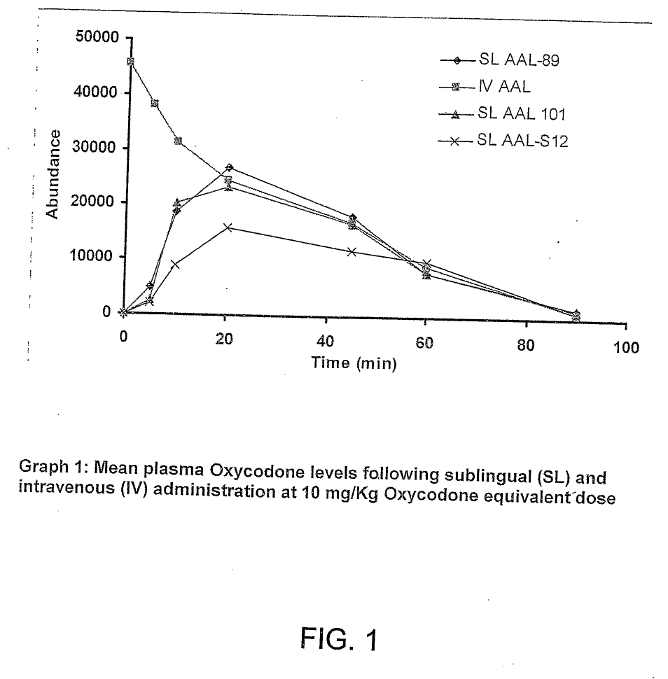

FIG. 1 illustrates the mean plasma oxycodone concentration versus time relationship that resulted after oxycodone formulations 1 and 2 were sprayed onto the sublingual mucosa of the rabbit, in comparison to intravenous (IV) administration of oxycodone. The concentration-time profiles were analyzed by a noncompartmental method, and the pharmacokinetic parameters were determined.

The mean AUC values for oxycodone after IV and after sublingual spray delivery of formulations 1 and 2 administration were 12791 ng.min/mL, 5807 ng.min/mL and 8965.3 ng.min/mL, respectively. The pharmacokinetic parameters of the two formulations in comparison with the intravenous route are presented in (Table 3).

TABLE 3
Pharmacokinetic parameters following intravenous administration
(IV) and sublingual spray administration of formulation 1 (pH
4) and formulation 2 (pH 9) to rabbits (n = 3).

| Parameter average | Intravenous | Sublingual | Sublingual |
|---|---|---|---|
| and Unit | (IV) | (pH 4) | (pH 9) |
| Cₘₐₓ (ng/mL) | 185.0 | 64.88 | 95.24 |
| tₘₐₓ (hr) | 0 | 0.33 | 0.33 |
| t_{1/2} (hr) | 1.13 | 1.00 | 1.11 |
| AUC_{∞} (ng · min/mL) | 12791 | 5807.0 | 8965.3 |
| AUC/AUC_{IV} | 1.00 | 0.454 ± 0.020 | 0.701 ± 0.179 |

Following sublingual spray administration, the bioavailabilities of formulation 1 and formulation 2 were obtained by comparing the mean AUC after intravenous and sublingual administration and were found to be 45.4±20.1%, and 70.1±17.9%, respectively (FIG. 1). Two-tailed t-test analyses indicated that there was no significant difference in oxycodone sublingual bioavailability between the two different pH formulations (p<0.05). All sublingual studies regardless of formulation pH, tₘₐₓ occurred around 20 minutes of spray solution application to the rabbit sublingual mucosa (FIG. 1).

The times were determined in reaching maximum plasma concentrations (20 minutes), as compared to immediate release oral tablets (1.3 hours), intramuscular (1.0 hour), and intranasal oxycodone (0.42 hour) in healthy volunteers (Eija Kalso, Journal of Pain and Symptom management. (2005); 29:S47-S56). The short sublingual tₘₐₓ value, which is similar to that obtained after nasal administration, may be due to a reduction in dose swallowing (i.e., reduction of gastrointestinal absorption) by introduction of the dose as a formulated solution through a spray pump device designed to deliver a 0.1 mL dose of the drug. The small droplet size increases the surface area of contact with the mucosal membrane, thus enhancing drug absorption profiles (Pankaj Modi et al., Diabetes/Metabolism Research and Reviews. (2002);18:S38-S42). Furthermore, Katz and Barr (Martin Katz et al., Journal of the American Pharmaceutical Association. (1955);44:476-480) have reported that a formulation solution with an alcoholic component is expected to alter the sublingual absorption profile of a drug, and that the use of the free base or the salt form of a drug with a specific vehicle had a more important effect on sublingual absorption than in oral or subcutaneous delivery.

The characteristics of sublingual absorption of drugs are dependent on both pH and lipid solubility (David S. Weinberg et al., Clin Pharmacol Ther. (1988);335-342; and L. S. Schanker, Pharmacol Rev. (1962);14:501-530). Sublingual bioavailability and absorption may be improved by converting the ionized form of oxycodone to the unionized form (free base) by adjusting the formulation pH. Thus, it was hypothesized that a low degree of ionization might favor maximal absorption. The analgesic, (oxycodone), is a weak base (pKa=8.53), with a partition coefficient equal to 0.7 (Eija Kalso, Journal of Pain and Symptom management. (2005); 29:S47-S56). Applying the Henderson-Hasselbalch equation, the predominant species in the formulation at pH 9.0 (formula 2) will be the unionized oxycodone free base, which will exist in equilibrium with its protonated form (Equation 2), whereas the protonated oxycodone species will be the more predominant form in the formulation at pH 4.0 (formula 1). In this respect, an increase in oxycodone bioavailability was observed with formulation 2 (pH 9.0) compared to formulation 1 (pH 4.0); however, the difference observed was not statistically significant (p<0.05). Thus, it is likely that the very low lipid solubility of both ionized and unionized oxycodone is the factor contributing most to the limited increase in bioavailability. Other factors which may be contributory are the low dose volume and the weak buffer capacity of the formulations once they are applied to the sublingual mucosal membranes.

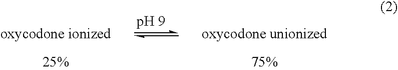

In adults, the bioavailability of oral, nasal and rectal oxycodone has been reported to be 40-60% when compared with parenteral administration (A. Takala et al., Acta Anaesthesiol Scand. (1997);41:309-312; and K. P. Leow et al., Therapeutic drug monitoring. (1992);14:479-484). Buccal and intranasal administration of oxycodone hydrochloride both afford similar bioavailability of around 55% (A. Takala et al., Acta Anaesthesiol Scand. (1997);41:309-312; and Hao Zhang, Clinical Pharmacokinetics. 2002);41:661-680). This is in close agreement with the sublingual bioavailability of 45.4±20.1% obtained in this study.

Further, the bioavailability of oxycodone was increased to 70.1±17.9%, when the formulation pH rose up to 9.0. However, this difference in bioavailability was found to be statistically insignificant (p<0.05). The terminal half-life of oxycodone after intravenous administration was close to the terminal half-life after sublingual administration. This result implies that there is no long-term deposition of oxycodone in the sublingual mucosa after transmucosal delivery. By-passing first pass metabolism of oxycodone by the gastrointestinal tract and the liver by administering the drug via sublingual spray enhanced oxycodone bioavailability. Also, a sublingual formulation of oxycodone permits the use of lower doses of oxycodone for pain management, because a greater portion of the medication would be absorbed directly into the bloodstream. This allows a direct route to the afflicted target area.

While the present invention has been described with reference to specific embodiments, this application is intended to cover those various changes and substitutions that may be made by those of ordinary skill in the art without departing from the spirit and scope of the appended claims.

What is claimed is:

1. A pharmaceutical composition comprising oxycodone or a pharmaceutically acceptable salt thereof and a pharmaceutically acceptable carrier, wherein the oxycodone or pharmaceutically acceptable salt thereof is provided in a form suitable for sublingual administration.

2. A liquid spray formulation, comprising:
(i) oxycodone or pharmaceutically acceptable salt or free base thereof,
(ii) buffered water; and
(iii) a polar organic solvent,
wherein the said polar organic solvent is present in an amount sufficient to enhance the solubility of the oxycodone free base or salt thereof in the water.

3. The formulation of claim 2, wherein the oxycodone is present as the free base or salt.

4. The formulation of claim 2, wherein the formulation is partially pressurized.

5. The formulation of claim 2, wherein the oxycodone or pharmaceutically acceptable salt or free base thereof, is present at a concentration of 0.1-10 mg/ml.

6. The formulation of claim 2, wherein the polar organic solvent is an alcohol.

7. The formulation of claim 6, wherein the alcohol is selected from the group consisting of ethanol, propylene glycol, glycerol, polyethylene glycol and mixtures thereof.

8. The formulation of claim 7, wherein the alcohol is ethanol.

9. The formulation of claim 2, wherein the polar organic solvent is present in an amount of 3-50% w/w.

10. The formulation of claim 2, wherein the formulation is buffered.

11. The formulation of claim 2, further comprising a sweetener.

12. The formulation of claim 11, wherein the sweetener is mannitol.

13. The formulation of claim 11, wherein the sweetener is saccharin or saccharin sodium.

14. The formulation of claim 2, further comprising a flavoring agent.

15. The formulation of claim 14, wherein the flavoring agent is menthol.

16. The formulation of claim 2, further comprising a penetration enhancer.

17. The formulation of claim 16, wherein the penetration enhancer is chitosan.

18. The formulation of claim 2, further comprising a mucoadherant.

19. The formulation of claim 18, wherein the mucoadherant is selected from the group consisting of chitosan, polyvinyl pyrrolidone, and gelatin.

20. The formulation of claim 2, wherein the formulation is suitable for sublingual administration.

21. The formulation of claim 2, wherein the formulation further comprises another active ingredient selected from the group consisting of an opioid analgesic, a non-opioid analgesic, an antidepressant, a compound which reacts with the NMDA receptor, a compound which reacts with the nicotinic receptor, and pharmaceutically acceptable salts thereof.

22. The formulation of claim 21, wherein the opioid analgesic is selected from the group consisting of codeine, morphine, hydromorphone, methadone, meperidine, levorphanol, fentanyl, meperidine, butorphanol, and pharmaceutically acceptable salts thereof.

23. The formulation of claim 21, wherein the non-opioid analgesic is selected from the group consisting of acetylsalicyclic acid, acetaminophen, ibuprofen, and pharmaceutically acceptable salts thereof.

24. The formulation of claim 21, wherein the antidepressant is selected from the group consisting of amitryptiline, desipramine, doxepin, imipramine, and pharmaceutically acceptable salts thereof.

25. The formulation of claim 21, wherein the compound which reacts with the NMDA receptor is selected from the group consisting of ketamine, norketamine, and pharmaceutically acceptable salts thereof.

26. The formulation of claim 21, wherein the compound which reacts with the nicotinic receptor is selected from the group consisting of nicotine, nornicotine, and pharmaceutically acceptable salts thereof.

27. A liquid spray formulation, comprising:
(i) oxycodone or pharmaceutically acceptable salt or free base thereof, in an amount of 0.01 mg/kg to about 10 mg/kg, wherein the liquid spray formulation is present in the amount of 100 mL.

28. A method of providing fast relief from pain, comprising administering to a subject in need thereof a pharmaceutically effective amount of oxycodone, by spraying the oxycodone onto the subject's sublingual mucosa.

29. The method of claim 28, wherein the pain is acute.

30. The method of claim 29, wherein the acute pain is post-operative pain, post-traumatic pain, or pain associated with cancer.

31. The method of claim 28, wherein the oxycodone is in the form of oxycodone free base or hydrobromide salt, or any acceptable salt dissolved in an ethanolic solution.

32. A method of providing fast relief from acute pain comprising administering to a subject in need thereof the formulation of claim 2, by spraying the formulation onto the subject's sublingual mucosa.

33. The method of claim 32, wherein relief from pain is achieved within 20 minutes.

34. The method of claim 32, wherein relief from pain is achieved within 5 minutes.

35. A metered dose dispensing system for the administration of the formulation of claim 2, comprising a sealed container fitted with a metering pump, an actuator and a channeling device.

36. The metered dose dispensing system of claim 35, containing a metering chamber which is adapted for dispensation with the container in the upright orientation, and wherein the metering chamber is in communication with the formulation by means of a dip-tube.

37. A liquid spray formulation, comprising:
(i) a compound that reacts with the same receptor as oxycodone or pharmaceutically acceptable salt or free base thereof,
(ii) buffered water; and
(iii) a polar organic solvent,
wherein the said polar organic solvent is present in an amount sufficient to enhance the solubility of the compound of step (i), free base or salt thereof in the water.
